SEQUENCE LISTING (8 sequences)

SEQ ID NO: 1 - protein, 111 aa, Ganoderma tsugae
  PEPTIDE (1)..(111)
MSDTALIFRLAWDVKKLSFDYTPNWGRGNPNNFIDTVTFPKVLTDKAYTYRVAVSGRNLGVKPSYAVESDGSQKVNFLEYNSGYGIADTNTIQVFVVDPDTNNDFIIAQWN

SEQ ID NO: 2 - protein, 114 aa, Flammulina Velutipes
  PEPTIDE (1)..(114)
SATSLTFQLAYLVKKIDFDYTPNWGRGTPSSYIDNLTFPKVLTDKKYSYRVVVNGSDLGVESNFAVTPSGGQTINFLQYNKGYGVADTKTIQVFVVIPDTGNSEEYIIAEWKKT

SEQ ID NO: 3 - protein, 112 aa, Volvariella volvacea
  PEPTIDE (1)..(112)
STDLTQLLFFIAYNLQKVNFDYTPQWQRGNPSSYIDAVVFPRVLTNKAYQYRVVTGDKDLGIKPSYSVQADGSQKVNLLEYNGGYGVADTTTIKIYVVDPSNGNQYLIAQWK

SEQ ID NO: 4 - protein, 111 aa, Ganoderma lucidum; identical to SEQ ID NO: 1
  PEPTIDE (1)..(111)

SEQ ID NO: 5 - protein, 111 aa, Ganoderma japoncium
  PEPTIDE (1)..(111)
MSDTALIFRLAWDVKKLSFDYTPTWGRGNPSRFVDNVTFPQVLADKAYTYRVVVSGRDLGVRPSYAVGSDGSQKVNFLEYNQGYGIADTNTIQVFVIDPDTDADFIIAQWN

SEQ ID NO: 6 - protein, 111 aa, Ganoderma microsporum
  PEPTIDE (1)..(111)
MSDTALIFTLAWNVKQLAFDYTPNWGRGRPSSFIDTVTFPTVLTDKAYTYRVVVSGKDLGVRPSYAVESDGSQKINFLEYNSGYGIADTNTIQVYVIDPDTGNNFIVAQWN

SEQ ID NO: 7 - protein, 111 aa, Ganoderma sinense
  PEPTIDE (1)..(111)
MSDTALIFRLAWDVKKLSFDYTPTWGRGNPSRFVDNVTFPQVLADKAYTYRVVVSGRDLGVRPSYAVGSDGSQKVNFLEYNQGYGIADTNTIQVFVIDPDTGADFIIAQWN

SEQ ID NO: 8 - protein, 114 aa, Nectria haematococca
  PEPTIDE (1)..(114)
MATTNDSAVLFYIVASQKKLSFDYTPNWGRGSPNSYIDNLTFPRVLTNKPYKYRVVKAGQDLGVRDSYSVQSDGSQKVNFLEYNAGRGIADTQTIQVYVVDPDNGNQYLVAQWK

(19) United States
(12) Patent Application Publication
(10) Pub. No.: US 2017/0368119 A1
(43) Pub. Date: Dec. 28, 2017

(54) METHODS AND COMPOSITIONS FOR TREATING C-MET ASSOCIATED CANCERS

(71) Applicant: YEASTERN BIOTECH CO., LTD, New Taipei City (TW)

(72) Inventors: Wen-Sheng Wu, Hualien (TW); Jia-Ru Wu, Hualien (TW); Chi-Tan Hu, Hualien (TW); Ren-In You, Hualien (TW); Pei-Ling Ma, Hualien (TW); Tzu-Chih Chen, New Taipei City (TW)

(73) Assignee: YEASTERN BIOTECH CO., LTD, New Taipei City, TW (TW)

(21) Appl. No.: 15/508,126

(22) PCT Filed: Aug. 28, 2015
(86) PCT No.: PCT/CN2015/088356

§ 371 Date: Mar. 2, 2017

Related U.S. Application Data
(60) Provisional application No. 62/044,415, filed on Sep. 2, 2014.

(51) Int. Cl. A61K 36/064; A61K 38/16; A61K 35/74

(52) U.S. Cl. CPC A61K 36/064; A61K 35/74; A61K 38/168

(57) ABSTRACT

The present invention provides the use of a fungal immunomodulatory protein (FIP) in manufacturing a medicament for inhibiting hepatocyte growth factor receptor (HGFR) activity in a cell, and for treating HGFR-associated cancer.

### CROSS-REFERENCE TO RELATED APPLICATIONS

This is a §371 National Stage application of International Application No. PCT/CN2015/088356 filed on 28 Aug. 2015, claiming the priority of U.S. Patent Application No. 62/044,415 filed on Sep. 2, 2014, the entireties of which are incorporated herein by references.

This application contains a Sequence Listing in computer readable form. The computer readable form is incorporated herein by reference.

### BACKGROUND OF THE INVENTION

### Field of the Invention

The invention relates to the use of a fungal immunomodulatory protein (FIP) in the treatment of cancers, more particularly of c-Met-associated cancers, still more particularly of hepatocellular carcinoma, to the use of an FIP in inhibiting tumor growth, migration, metastasis or recurrence of cancers, more particularly of c-Met-associated cancers, still more particularly of hepatocellular carcinoma. The invention also relates to methods and compositions for blocking c-Met signaling.

### Description of Prior Art

Hepatocellular carcinoma (HCC) is one of the most devastating cancers worldwide, especially in Southeast Asia. The poor prognosis and recurrence of HCC were caused by tumor metastasis. Recently, the tumor microenvironment was highlighted to be critical in triggering tumor metastasis. The tumor microenvironment comprises the primary tumor itself which may interact with stromal and inflammatory cells leading to secretion of a lot of metastatic growth factors including hepatocyte growth factor (HGF) triggering metastatic phenotypical change of primary tumor.

It is known in that art that activation of c-Met may occur in HCCs in an autocrine fashion as evidenced by high level of intracytoplasmic HGF. Moreover, high HGF level in serum and deregulated expression of c-Met in HCCs were closely associated with early recurrence and patients with a high-Met expressing HCCs usually have shorter 5-year survival after curative surgical resection. In addition, a group of HCCs (27%) with c-Met-induced transcriptional signature was characterized by a higher rate of vascular invasion. On the other hand, in vitro studies also demonstrated the effects of HGF on metastatic changes of HCC including EMT, migration and invasion. Therefore, HGF-c-Met signaling is regarded as the most promising therapeutic target for prevention of HCC progression.

Binding of HGF to c-Met induces autophosphorylation of its cytoplasmic domain, followed by recruiting upstream regulators such as Gab, Grb2, and PI3K, activating downstream signaling including extracellular signal-regulated protein kinase (ERK), c-Jun kinase (INK) and protein kinase B (AKT). In the past decade, a lot of small molecules were designed for blocking c-Met signal cascade. Up to now, at least 17 inhibitors including JNJ-38877605, GEN-203, and ARQ197 are under clinical evaluation (Zhu K et al., Expert Opin Ther Pat 2014, 24:217-230).

The toxicities of c-Met inhibitors have been demonstrated in a lot of studies. In one animal experiment, GEN-203 may cause significant liver and bone marrow toxicity. In the clinical trials, severe adverse events including anemia and neutropenia caused by ARQ 197 for treatment of HCC were observed. These side effects may be ascribed to broad expression of c-Met in epithelial cells of many organs and its critical biological functions, such as defensive response to tissue damage.

Although being engaged with potential challenges as described above, there are still great potentials for improving c-Met therapeutic approach. Firstly, large scale screening of HCC with active c-Met signaling can be performed to enroll suitable patients for the trials. Secondly, more effective c-Met inhibitors that warrant safety should be carefully selected. Thirdly, patient derived HCC can be established for testing the efficiency of the HCC antagonist.

Several proteins from edible fungi, such as Ganoderma Lucidium (Ling zhi or Reishi), Volvariella Volvacea (Chinese Mushroom), Flammulina Velutipes (Golden needle mushroom), share similar amino acid sequences and immunomodulatory functions. These proteins were named fungal immunomodulatory proteins (FIPs) (Ko J. L., Eur J. Biochem. 1995; 228:244-249).

At least ten FIPs have been identified and isolated from Ganoderma lucidum, Flammulina veltipes, Volvariella volvacea, Ganoderma tsugae, Ganoderma japoncium, Ganoderma microsporum, Ganoderma sinense and Nectria haematococca, Tremella fuciformis, Antrodia camphorate and designated LZ-8 (also known as FIP-glu), FIP-fve, FIP-vvo, FIP-gts, FIP-gja (GenBank: AY987805), FIP-gmi, FIP-gsi, FIP-nha, FIP-tfu (GenBank: EF152774), FIP-aca, respectively (Hsu H C, et al., Biochem J 1997; 323 (Pt 2):557-565; Kong et al., Int. J. Mol. Sci. 2013; 14: 2230-2241; Han et al., J Appl Microbiol 2010, 109:1838-44; and China Patent No. CN102241751B). Among them, the DNA sequence of FIP-gts was found to be identical to the sequence of LZ-8 in Ganoderma lucidium. Both proteins exhibited the same immunoactivity, indicating that they are the same protein. FIPs are mitogenic in vitro for human peripheral blood lymphocytes (hPBLs) and mouse splenocytes. They induce a bell-shaped dose-responsive curve similar to that of lectin mitogens. Activation of hPBLs with FIPs results in the increased production of molecules of IL-2, IFN-γ and tumor necrosis factor-α associated with ICAM-1 expression (Wang P H, et al., J Agric Food Chem 2004; 52(9):2721-2725.). FIPs can also act as immunosuppressive agents. In vivo these proteins can prevent systemic anaphylactic reactions and significantly decrease footpad edema during Arthus reaction in mouse. These observations suggest that FIPs are both health promoting and therapeutic.

Analyzing the secondary structure with Gamier analysis, FIP-gts was predicted to have two α-helices, seven β-sheets and one β-turn. The molecular weight of FIP-gts was determined to be 13 kD using SDS-PAGE analysis. Connecting the amino acids with 20 μM glutaraldegyde (protein conjugate), FIP-gts was found to form a 26 kD homodimer.

Although the immunomodulatory activity of FIPs has been studied extensively, their anticancer activity has not been investigated until recent years. U.S. Pat. No. 8,629,096, assigned to the Applicant, discloses that FIP-gts demonstrates anti-proliferative activity on breast cancer, lung cancer, prostate cancer and melanoma, suggesting the potential utility of FIPs as a broad-spectrum anticancer agent. FIP-gts was also found to exhibit lethal effect on a gastric cancer cell line (Liang C et al., Oncol Rep 2012, 27:1079-1089). U.S. Patent Publication No. 2011/009597 provides additional experimental data showing the capability of a recombinant FIP-gts protein expressed from Pichia yeast in inducing the programmed cell death of leukemia cells in vitro and suppressing the growth of hepatoma cells in vivo. U.S. Pat. No. 8,476,238 teaches that FIP-gmi may inhibit invasion and metastasis of lung cancer via inhibiting epidermal growth factor receptor (EGFR) signaling. Additional in vitro studies reported that FIP-gts may also suppress cell migration of cervical cancer (Wang P H et al., Reprod Sci 2007, 14:475-485). However, the anti-metastatic effects of FIPs on cancers remain to be studied further.

The molecular mechanisms for anti-tumor activity of FIP-gts have been preliminarily studied. FIP-gts may stabilize p53 for increasing CDK inhibitor p21 for cell cycle arrest of lung cancer. U.S. Patent Publication No. 2007/071766, assigned to the Applicant, disclosed that FIP-gts can repress telomerase activity in lung adenocarcinoma cells. On the signal transduction level, FIP-gts may influence protein kinase C (PKC)/ROS, PTK/PLC/PKCalpha/ERK1/2, and PTK/PLC/PKCalpha/p38 cascade. Among these, PKC and ERK were known to be essential for HGF/c-Met signaling. Until now, whether any FIP may block c-Met dependent signaling has not been investigated.

### SUMMARY OF THE INVENTION

In the first aspect provided herein is a method for inhibiting hepatocyte growth factor receptor (c-Met) activity in a cell, comprising contacting an effective amount of a fungal immunomodulatory protein (FIP) with the cell to inhibit c-Met activity.

In the second aspect provided herein is fungal immunomodulatory protein (FIP) for use in inhibiting hepatocyte growth factor receptor (c-Met) activity in a cell.

In the third aspect provided herein is a composition for inhibiting hepatocyte growth factor receptor (c-Met) activity in a cell, comprising an effective amount of a fungal immunomodulatory protein (FIP) to inhibit c-Met activity.

In the fourth aspect provided herein is a method for inhibiting metastasis of a hepatocyte growth factor receptor (HGFR)-associated cancer in a subject, comprising administering to said subject an effective amount of a fungal immunomodulatory protein (FIP) to inhibit metastasis of the HGFR-associated cancer.

In the fifth aspect provided herein is fungal immunomodulatory protein (FIP) for use in inhibiting metastasis of a hepatocyte growth factor receptor-associated cancer in a subject.

In the sixth aspect provided herein is a pharmaceutical composition for inhibiting metastasis of a hepatocyte growth factor receptor (HGFR)-associated cancer, comprising an effective amount of a fungal immunomodulatory protein (FIP) to inhibit metastasis of the HGFR-associated cancer in a subject.

In the seventh aspect provided herein is a method for inhibiting cell migration of a hepatocyte growth factor receptor-associated cancer in a subject, comprising administering to said subject an effective amount of a fungal immunomodulatory protein (FIP).

In the eighth aspect provided herein is fungal immunomodulatory protein (FIP) for use in inhibiting cell migration of a hepatocyte growth factor receptor-associated cancer in a subject.

In the ninth aspect provided herein is a composition for inhibiting cell migration of a hepatocyte growth factor receptor-associated cancer in a subject, comprising an effective amount of a fungal immunomodulatory protein (FIP).

In the tenth aspect provided herein is a method for treating a cancer by blocking hepatocyte growth factor receptor (c-Met) signaling in a subject in need thereof, comprising administering to the subject an effective amount of a fungal immunomodulatory protein (FIP) to block the c-Met signaling.

In the eleventh aspect provided herein is fungal immunomodulatory protein (FIP) for use in treating a cancer by blocking hepatocyte growth factor receptor (c-Met) signaling in a subject in need thereof.

In the twelfth aspect provided herein is a pharmaceutical composition for treating a cancer by blocking hepatocyte growth factor receptor (c-Met) signaling in a subject in need thereof, comprising an effective amount of a fungal immunomodulatory protein (FIP) to block the c-Met signaling.

In the thirteenth aspect provided herein is a method for preventing recurrence of a hepatocyte growth factor receptor-associated cancer in a subject, comprising administering to said subject an effective amount of a fungal immunomodulatory protein (FIP) to prevent recurrence of the HGFR-associated cancer.

In the fourteenth aspect provided herein is fungal immunomodulatory protein (FIP) for use in preventing recurrence of a hepatocyte growth factor receptor-associated cancer in a subject.

In the fifteenth aspect provided herein is a pharmaceutical composition for preventing recurrence of a hepatocyte growth factor receptor (HGFR)-associated cancer, comprising an effective amount of a fungal immunomodulatory protein (FIP) to prevent recurrence of the HGFR-associated cancer.

In the sixteenth aspect provided herein is a method for inhibiting metastasis of hepatocellular carcinoma in a subject, comprising administering to said subject an effective amount of a fungal immunomodulatory protein (FIP) to inhibit metastasis of hepatocellular carcinoma.

In the seventeenth aspect provided herein is fungal immunomodulatory protein (FIP) for use in inhibiting metastasis of hepatocellular carcinoma in a subject.

In the eighteenth aspect provided herein is a pharmaceutical composition for inhibiting metastasis of hepatocellular carcinoma in a subject, comprising an effective amount of a fungal immunomodulatory protein (FIP) to inhibit metastasis of hepatocellular carcinoma.

In the nineteenth aspect provided herein is a method for inhibiting cell migration of hepatocellular carcinoma in a subject, comprising administering to said subject an effective amount of a fungal immunomodulatory protein (FIP) to inhibit cell migration of hepatocellular carcinoma.

In the twentieth aspect provided herein is fungal immunomodulatory protein (FIP) for use in inhibiting cell migration of hepatocellular carcinoma in a subject.

In the twenty-first aspect provided herein is a composition for inhibiting cell migration of hepatocellular carcinoma in a subject, comprising an effective amount of a fungal immunomodulatory protein (FIP) to inhibit cell migration of hepatocellular carcinoma.

In the twenty-second aspect provided herein is a method for preventing recurrence of hepatocellular carcinoma in a subject, comprising administering to said subject an effective amount of a fungal immunomodulatory protein (FIP) to prevent recurrence of hepatocellular carcinoma.

In the twenty-third aspect provided herein is fungal immunomodulatory protein (FIP) for use in preventing recurrence of hepatocellular carcinoma in a subject.

In the twenty-fourth aspect provided herein is a pharmaceutical composition for preventing recurrence of hepatocellular carcinoma in a subject, comprising an effective amount of a fungal immunomodulatory protein (FIP) to prevent recurrence of hepatocellular carcinoma.

### BRIEF DESCRIPTION OF THE DRAWINGS

The above and other objects, features and effects of the invention will become apparent with reference to the following description of the preferred embodiments taken in conjunction with the accompanying drawings, in which:

FIGS. 1A and 1B show the morphology and motility of indicated HCC cell lines by performing a wound healing assay for 48 hours, where pictures were taken under phase contrast microscopy (200× magnification);

FIG. 1C shows the quantitative analysis of motility of indicated HCCs, where relative motility of each HCC was calculated by taking motility of HCC340 as 1.0, and (**) and (*) represent statistical significance (p<0.005 and p<0.05, respectively, n=4) for differences of motility between the indicated HCCs and the HCC340;

FIG. 2 shows an immuno-blotting analysis comparing various signaling molecules in HCC cell lines using GAPDH as loading control, in which the data were representative of 2 reproducible experiments;

FIGS. 3A, 3B and 3C show the suppressive effects of FIP-gts and JNJ on cell migration of HCC329 and HCC372 and HGF-induced cell migration of HepG2;

FIG. 4 are gross photographs of whole liver specimens taken from the SCID mice, demonstrating the suppressive effects of FIP-gts and JNJ on tumor growth and metastasis of HCC329 in a SCID mice model, in which intra-hepatic metastases were shown in the medium- and JNJ-treated mice groups but not in the FIP-gts-treated mice group;

FIGS. 5A-5D show that FIP-gts block signaling transduction in c-Met positive and negative HCCs and HGF-induced HepG2;

FIGS. 6A-6B are histograms showing the effects of FIP-gmi, FIP-fve and FIP-vvo on cell viability of c-Met positive and negative HCCs;

FIG. 7 shows histological images of HCC cells in the transwell migration assay, showing that FIP-gmi, FIP-fve and FIP-vvo suppress cell migration of HCC329 and HCC372; and

FIG. 8 shows another immuno-blotting analysis comparing various signaling molecules in HCC cell lines using GAPDH as loading control.

### DETAILED DESCRIPTION OF THE INVENTION

The present invention is based on the discovery that the fungal immunomodulatory protein (FIP), such as FIP-gts, is effective in inhibiting c-Met activity, indicating that the migration and metastasis of the cancer cells which express c-Met protein can be suppressed by administration of the FIP to inhibit the c-Met activity. In particular, when FIP-gts was used as an exemplary embodiment of the FIP, the inhibitory effect thereof on c-Met activity was found more potent compared to a known c-Met inhibitor, JNJ-38877605. The FIP can also block constitutive c-Met-dependent signaling more effectively than JNJ-38877605. The invention further found that HGF-induced c-Met-dependent signaling and migration of cancer cells can be inhibited by the FIP. These findings suggest that an FIP is therapeutically useful in treatment of proliferative disorders, such as cancers, including cancers associated with c-Met signaling or c-Met-associated cancers. Especially, treatment of a cancer cell that expresses c-Met protein with an FIP can result in blocking the c-Met signaling and inhibiting downstream events related to the c-Met signaling, such as activation of JNK and ERK in the cancer cell. The invention further found that, in addition to suppressing constitutive and HGF-induced c-Met signaling, the FIP may also block constitutive c-Met independent signaling in HCCs and inhibit the growth, migration and metastasis of the HCC cells in which c-Met protein is substantially unexpressed or the active form of c-Met protein is substantially undetectable.

c-Met, also known as hepatocyte growth factor receptor (HGFR), is encoded by Met proto-oncogene and possesses tyrosine-kinase activity. Hepatocyte growth factor (HGF) is the only known natural ligand of the Met receptor. HGF/c-Met signaling, also interchangeably referred to herein as HGFR signaling, triggers an invasive growth program that is thought to be essential in early embryonic development but, when dysregulated, can result in malignant growth, motility, migration and invasion of abnormal cells. It is believed that HGFR signaling, including MAPK (mitogen activated protein kinase)-, PI3K/AKT-, STAT3- and β-catenin-related pathways, plays an important role in the development of cancer through induction of uncontrolled cell growth and angiogenesis; and is responsible for triggering tumor progression via induction of scattering, namely, cell dissociation (also called epithelial mesenchymal transition or EMT), and migration and invasion due to metalloprotease production, which often leads to metastasis.

Activation of HGFR signaling is mainly dependent on phosphorylation of certain amino acid residues on the Met receptor, in which Tyr1234 and Tyr1235 in the kinase domain are auto-phosphorylated in response to HGF binding, leading to further phosphorylation of Tyr1349 and Tyr1356 in the C-terminal multifunctional docking site. The phosphorylation of HGFR in turn activates non-receptor tyrosine kinase Src, focal adhesion kinase (FAK) and Ras/MEK/ERK and PI3K/AKT signaling cascades. Therefore, the terms “inhibiting HGFR activity,” “suppressing HGFR activation” and “blocking HGFR signaling” are used interchangeably herein to mean preventing the activation of HGFR and/or reducing the kinase activity of HGFR by administering an FIP or any c-Met inhibitor, such as JNJ-38877605, as measured by, for example, a detectable decrease in the overall expression level of HGFR protein, the phosphorylated level thereof (especially the phosphorylated level of HGFR at Tyr1234) or the phosphorylated level of a downstream effector thereof, such as ERK and JNK, compared to that measured in the absence of the FIP or the c-Met inhibitor.

Constitutive activation and aberrant expression of HGFR may be ligand-independent and are believed to contribute to tumor growth, migration, invasion and metastasis in numerous human cancers (Mizuno S. and Nakamura T., Int. J. Mol. Sci. 2013, 14:888-919). As used herein, the term “HGFR-associated cancer” or “HGFR-positive cancer” refers to a cancer which expresses HGFR protein on surfaces of the cancer cells and typically has aberrant expression and/or activation of HGFR protein that may cause, enhance or contribute to growth, migration, metastasis or recurrence of the cancer. In an HGFR-associated cancer, the presence of HGFR protein and its phosphorylated forms can be readily detected by any conventional techniques for quantitative or qualitative analysis of a peptide or a protein, such as immunoblotting, enzyme-linked immunosorbent assay (ELISA), immuno-precipitation and fluorescence microscopy. In some embodiments, the cancer is a solid tumor selected from the group consisting of hepatocellular carcinoma, hereditary and sporadic human papillary renal carcinomas, ovarian cancer, prostate carcinoma, gallbladder carcinoma, breast carcinoma, melanoma, glioblastoma, head-and-neck squamous carcinoma, esophageal cancer, gastric cancer, pancreatic cancer, mesothelioma, colorectal cancer, and osteogenic sarcoma. In other embodiments, the cancer is a liquid tumor, e.g., lymphoma and multiple myeloma.

As disclosed herein, the present invention provides a method for inhibiting HGFR activity in a cancer cell, comprising contacting an effective amount of an FIP with HGFR. In some embodiments, the HGFR activity is dysregulated due to over-expression of HGF or HGFR, which means an upward deviation in levels of expression of HGF or HGFR as compared to the baseline expression level in non-cancerous tissue of the same type. In some embodiments, the HGFR is abnormally active in the cell, for example, abundantly present in its phosphorylated forms, especially those phosphorylated at Tyr1234. The term “cell,” as used herein, refers to a cell that is in vitro, ex vivo or in vivo. In some embodiments, an ex vivo cell can be part of a tissue sample excised from an organism, such as a mammal. In some embodiments, an in vitro cell can be a cell in a cell culture. In some embodiments, an in vivo cell is a cell living in an organism, such as a mammal. As used herein, the term “contacting” refers to the bringing together of indicated moieties in an in vitro system or an in vivo system. For example, “contacting” an FIP with an HGFR includes administration of the FIP to an individual or a patient, such as a human, as well as, for example, introducing an FIP into a sample containing a cell on which the HGFR is expressed.

In a preferred embodiment, the invention disclosed herein relates to using an FIP to inhibit cell migration or metastasis of a HGFR-associated cancer, such as those selected from the group consisting of hepatocellular carcinoma, hereditary and sporadic human papillary renal carcinomas, ovarian cancer, prostate carcinoma, gallbladder carcinoma, breast carcinoma, melanoma, glioblastoma, head-and-neck squamous carcinoma, esophageal cancer, gastric cancer, pancreatic cancer, colorectal cancer, and osteogenic sarcoma, lymphoma and multiple myeloma. Among the cancers listed above, hepatocellular carcinoma is the most preferred. According to the invention, the term “migration” refers to movement of a cell or cells from one locus to another. As used herein, the term “metastasis” refers to a process by which a cancer cell is translocated from a primary cancer site, where it initially formed from a normal, hyperplastic or dysplastic cell, to a secondary site where the translocated cancer cell lodges and proliferates. Typically, the metastasis process involves the ability of a cancer cell to migrate through a physiological barrier, such as basement membranes and other extracellular matrices known in the art.

The present invention further contemplates preventing recurrence of a HGFR-associated cancer, as micro-metastasis of a cancer often results in a risk of recurrence. Indeed, the recurrence of a cancer may be attributed to incomplete removal or killing of cells from the primary cancer and may occur locally (the same site of primary cancer), regionally (in vicinity of primary cancer, possibly in the lymph nodes or tissue) and/or distally as a result of metastasis. According to the Examples described below, the administration of an FIP was shown to induce regression of primary tumor and suppress intra-metastasis of HCC in liver, suggesting that the recurrence of a HGFR-associated cancer can be prevented effectively by the invention disclosed herein. Therefore, the terms “preventing recurrence” and “prevention of recurrence” as used herein refer to reducing or eliminating the return of a cancer after a remission.

In another preferred embodiment, the invention disclosed herein is suitable for cancer therapy. The term “treating” or “treatment” as used herein relates to the amelioration of the symptoms of the prevailing disease condition, deceleration of the course of disease, especially to the suppression of tumor growth, the inhibition of recurrence or metastasis of the malignancy, and the induction of tumor regression. In some embodiments, the term “treating” or “treatment” refers to the reduction or stabilization of tumor size or cancerous cell count. Desirably, the treated cancer is a HGFR-associated cancer, such as those selected from the group consisting of hereditary and sporadic human papillary renal carcinomas, ovarian cancer, gallbladder carcinoma, glioblastoma, head-and-neck squamous carcinoma, esophageal cancer, pancreatic cancer, colorectal cancer, and osteogenic sarcoma.

Nevertheless, previous studies indicated that c-Met over-expression was observed only in 20-48% of human HCC samples. This observation is somewhat confirmed by Example 3 disclosed herein, where only four out of ten cell lines under analysis were shown to have detectable levels of c-Met and p-c-Met. For those HCCs with negative c-Met signaling, c-Met targeting approach will not be adequate.

It was surprisingly found by the inventors that FIP can still suppress the cell migration, metastasis and recurrence of the cancers, especially the HCCs, which do not substantially express c-Met protein and/or phosphorylated c-Met, suggesting that FIP may suppress tumor progression by multiple mechanisms, including inhibiting c-Met dependent signaling pathway and at least one c-Met independent signaling pathway. The HCCs which do not substantially express c-Met protein and/or phosphorylated c-Met, also referred to herein as “HGFR-negative HCCs,” encompass those having no detectable level of c-Met and/or p-c-Met (especially those phosphorylated at Tyr1234) observed in the carcinoma cells as assessed by any conventional techniques for quantitative or qualitative analysis of a peptide or a protein, such as immunoblotting, enzyme-linked immunosorbent assay (ELISA), immuno-precipitation and fluorescence microscopy. It is quite likely that HGFR may not be crucial to the tumor progression of the HGFR-negative HCCs. As described in Example 7 below, EGFR signaling, but not c-Met signaling, is active in HGFR-negative HCCs and can be suppressed by the FIP disclosed herein.

The term “fungal immunomodulatory protein,” or abbreviated as “FIP,” is used herein to refer to a protein belonging to the protein family first defined in Ko et al., Eur J. Biochem. 1995; 228:244-249, based on the similarity in amino acid sequence and the effects on immunological response. It has been reported that the immunomodulatory proteins in the FIP family share a sequence homology of at least 57%. In particular, the primary structures of FIP-gts, FIP-fve, FIP-vvo and LZ-8 exhibit a high sequence homology of 60-70% (Kong et al., Int. J. Mol. Sci. 2013, 14, 2230-2241; China Patent No. CN102241751B; Wang X F et al., Curr Topics Nutraceutical Res. 2012, 10(1): 1-12; and Li O Z et al., Crit. Rev. Biotech. 2011, 31(4):365-375). Therefore, the term “fungal immunomodulatory protein” used herein is intended to encompass any polypeptide having an amino acid homology of at least 57%, preferably at least 60%, at least 70%, at least 80%, at least 90% or at least 95%, to the amino acid sequence of FIP-gts indicated by SEQ ID NO: 1 and having the ability to induce, enhance or extend the immune response in a subject. In one preferred embodiment, the fungal immunomodulatory protein has an amino acid sequence selected from the group consisting of SEQ ID NO: 1 (FIP-gts), SEQ ID NO: 2 (FIP-fve), SEQ ID NO: 3 (FIP-vvo), SEQ ID NO: 4 (LZ-8), SEQ ID NO: 5 (FIP-gja), SEQ ID NO: 6 (FIP-gmi), SEQ ID NO: 7 (FIP-gsi) and SEQ ID NO: 8 (FIP-nha), and functional variants thereof as an immunomodulatory agent. In another preferred embodiment, the FIP is derived from a Ganoderma species or Volvariella volvacea and, more preferably, has an amino acid sequence selected from the group consisting of SEQ ID NO: 1, SEQ ID NO: 2, SEQ ID NO: 3, SEQ ID NO: 4, SEQ ID NO: 5, SEQ ID NO: 6 and SEQ ID NO: 7. The most preferred is that comprising, consisting essentially of, or consisting of an amino acid sequence of SEQ ID NO: 1.

The FIP used herein may be obtained from a natural source, from a fungal culture or by recombinant expression in a prokaryotic or eukaryotic microorganism host, such as a bacterium or yeast host. The FIP thus obtained may be either in the form of a crude preparation or in a refined formulation separated, fractionated, or partially or substantially purified from the fungal matter by any suitable technique. Preferably the protein is at least partially purified before use. A useful process for preparing the FIP is disclosed in WO2005/040375A1, which involves culturing a yeast transformant harboring an expression vector carrying an FIP gene and harvesting a recombinant FIP protein from the yeast culture. Other useful methods may be found from, for example, Kong et al. (supra); CN101205553A; and Wang X F et al., Curr Topics Nutraceutical Res. 2012; 10(1): 1-12.

In the case where the FIP used is LZ-8 or FIP-gts, it can be either isolated directly from G. lucidum or G. tsugae, or prepared by recombinant protein technology in a host cell system. The host cells may be a yeast or bacterium system. Preferably, said host cell system is selected from the group consisting of Saccharomyces cerevisiae, Pichia pastoris, Hansenula polymorpha, Candida utilis, Candida boidinii, Candida maltosa, Kluyveromyces lactis, Yarrowia hpolytica, Schwanniomyces occidentalis, Schizosaccaromyces pombe, Torulopsis sp., Arxula adeninivorans, Aspergillus sp. (such as A. nidulans, A. niger, A. awamori, and A. oryzae), and Tricoderma sp. (such as T. reesei).

The FIPs used herein were substantially isolated or purified according to the methods described above, such as that disclosed in WO2005/040375A1. The term “substantially isolated or “substantially purified” as used herein refers to FIPs that are removed from their natural environment or host system, and are at least 60% free, preferably at least 75% free, and more preferably at least 90% free, even more preferably at least 95% free from other components with which they are naturally associated or with which they are accompanied in the host system.

As used herein, the term “subject” is intended to encompass human or non-human vertebrates, such as non-human mammal Non-human mammals include livestock animals, companion animals, laboratory animals, and non-human primates. Non-human subjects also include, without limitation, horses, cows, pigs, goats, dogs, cats, mice, rats, guinea pigs, gerbils, hamsters, mink, rabbits and fish. It is understood that the preferred subject is a human, especially a human patient afflicted with cancer or at risk for cancer, such as hepatocellular carcinoma.

For the purpose of research, the term “subject” may refer to a biological sample as defined herein, which includes but is not limited to a cell, tissue, or organ. Accordingly, the invention disclosed herein is intended to be applied in vivo as well as in vitro.

According to the invention, the term “administering to a subject” includes dispensing, delivering or applying the FIP in a suitable pharmaceutical formulation to a subject by any suitable route for delivery of the FIP to the desired location in the subject to contact the FIP with target cells or tissues.

The FIP may be administered to the subject by any suitable route, such as a topical, rectal, enteral or parenteral route, for example, an oral, intravenous, subcutaneous, intratumoral, intramuscular, intraperitoneal, transdermal, intrathecal, or intracerebral route. Administration can be either rapid as by injection, or over a period of time as by slow infusion or administration of a slow release formulation.

In one preferred embodiment, the FIP is to be administered orally and prepared in the form of an orally administrable formulation. Such formulation is preferably formulated with a suitable carrier, excipient, lubricant, emulsifying agent, suspending agent, sweetening agent, flavor agent, preserving agent and pressed as tablet or encapsulated as solid capsule or soft capsule. It is also contemplated that such formulation can be designed as following dosage forms, either oral solution, or oral sachet, or oral pellet. Or apart from being administered orally, it is contemplated that such formulations are designed as enema, or suppository, or implant, or patch, or cream, or ointment dosage forms. Some examples of suitable carriers, excipients, and diluents include lactose, dextrose, sucrose, sorbitol, mannitol, starches, gum acacia, calcium phosphate, alginates, calcium silicate, microcrystalline cellulose, polyvinylpyrrolidone, cellulose, gelatin, syrup, methyl cellulose, methyl- and propylhydroxybenzoates, talc, magnesium, stearate, water, mineral oil, and the like. The formulation can additionally include lubricating agents, wetting agents, emulsifying and suspending agents, preserving agents, sweetening agents or flavoring agents. The FIP composition may be formulated so as to provide rapid, sustained, or delayed release of the active ingredients after administration to the patient by employing procedures well known in the art. The formulation can also contain substances that diminish proteolytic, nucleic acid and other degradation and/or substances that promote absorption such as, for example, surface active agents. The composition may be complexed with polyethylene glycol (i.e., PEGylated), albumin or the like to help promote stability in the bloodstream.

Another preferred preparation for the FIP utilizes a vehicle of physiological saline solution; it is contemplated that other pharmaceutically acceptable carriers such as physiological concentrations of other non-toxic salts or compounds, 5% aqueous glucose solution, sterile water or the like may also be used. It may also be desirable that a suitable buffer be present in the composition. Such solutions can, if desired, be lyophilized and stored in a sterile ampoule ready for reconstitution by the addition of sterile water for ready injection. The primary solvent can be aqueous or alternatively non-aqueous.

The carrier can also contain other pharmaceutically-acceptable excipients for modifying or maintaining the pH, osmolarity, viscosity, clarity, color, sterility, stability, rate of dissolution, or odor of the formulation. Similarly, the carrier may contain still other pharmaceutically-acceptable excipients for modifying or maintaining release or absorption or penetration across the blood-brain barrier. Such excipients are those substances usually and customarily employed to formulate dosages for parenteral administration in either unit dosage or multi-dose form or for direct infusion by continuous or periodic infusion.

The FIP may conveniently be formulated in unit dosage form described above by conventional pharmaceutical techniques. Such techniques include the step of bringing into association the FIP and the physiologically acceptable carriers, diluents, adjuvants and/or excipient(s). In general, the formulations are prepared by uniformly and intimately bringing into association the FIP with liquid carriers or finely divided solid carriers or both, and then, if necessary, shaping the product.

The FIP is administered to the subject in a therapeutically effective amount to elicit the biological or medicinal response that is being sought in a cell, tissue, system, animal or human by a researcher, veterinarian, medical doctor or other clinician and preferably to stabilize, ameliorate or alleviate the symptom of the disease condition in the subject, such as decreasing the tumor growth, cell migration, metastasis or recurrence of a cancer and inducing tumor regression within the subject. Therefore, the term “effective amount” and the phrase “in an amount effective to” are interchangeably used herein and meant to refer to an amount of an FIP which produces a medicinal effect observed as reduction in the symptoms above when the effective amount of the FIP is administered to the subject. While the effective amounts are typically determined by the effect they have compared to the effect observed when a composition which includes no FIP described herein (i.e., a control) is administered to a similarly situated patient, the actual dose is calculated dependent upon the particular route of administration selected. The actual dose may be calculated dependent upon the particular route of administration selected. Further refinement of the calculations necessary to determine the appropriate dosage for administration is routinely made by those of ordinary skill in the art. Thus, when administered to a human subject, the FIP is preferably administered daily, weekly or twice a week, at an amount ranging from 0.01 mg/kg body weight/day to 100 mg/kg body weight/day, more preferably from 0.1 mg/kg/day to 10 mg/kg/day. Dose administration can be repeated depending upon the pharmacokinetic parameters of the dosage formulation and the route of administration used.

### EXAMPLES

The following examples are given for the purpose of illustration only and are not intended to limit the scope of the invention. It should be noted that, in the examples described below, paired Student's t test was done to statistically analyze the differences of band intensities on Western blot and quantitative estimation of cell motility between the indicated groups. Quantitative data were expressed as mean±coefficient variation (C.V.) as shown by the error bars in each figure. Reproducibility of the indicated molecules on immunohistochemical images was statistically analyzed using the Fisher Exact v2 test for categorical data.

### Example 1: Establishment of Patient-Derived HCC for Preclinical Trial of FIP-gts

Clinically derived HCC cell lines were established from parts of HCC tissues obtained from surgery with patient's consents and approved by Buddhist Tzu Chi General Hospital Research Ethic Committee (IRB 101-62). Briefly, HCC tissues were pretreated with collagenase followed by selection of HCC cell lines on mitomycin-treated NIH3T3 feeder layer for 4-6 passages. Homogenous HCC cell populations were obtained and tested for their sustained proliferation ability (over 20 passages) and metastatic potentials both in vitro and in vivo. The characteristics of the HCC tumor cell lines were validated by detecting the HCC tumor makers such as Glypican 3 (GCP3) after over 40 passages.

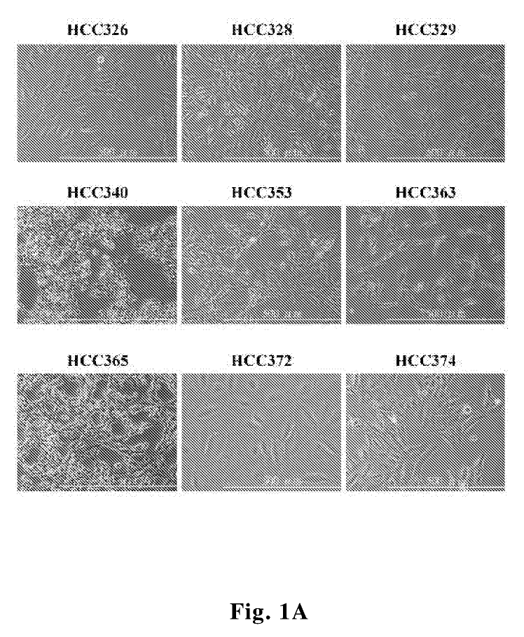

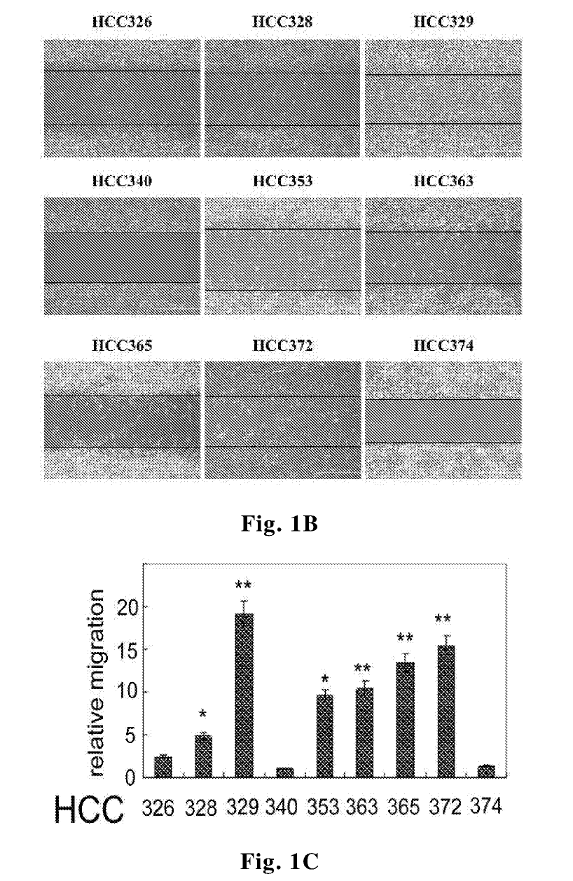

Patient-derived HCC cell lines were established and their phenotypes were characterized. Morphology of 9 HCCs (denoted as HCC329, 328, 326 and 340, 353, 365, 363, 372, 274, respectively) were demonstrated (FIG. 1A). Some of the cell lines (HCC329, 353, 365, 363 and 372) exhibited mesenchymal phenotype, whereas the others (such as HCC340, 374) exhibited epithelial type.

### Example 2: Wound Healing Migration Assay

The HCC cells were cultivated on 24-well plates accompanied with a wound healing culture insert until confluence followed by serum starvation of the cells for 24 hours and the culture insert were then removed. After appropriate treatments, pictures were taken under a phase contrast microscope at indicated times. Quantitative analysis of motility of the HCC cell lines described in Example 1 was performed by directly counting the cells migrating into the blanking area at 48 hours using Image J. Software available from the NIH website. The results were shown in FIG. 1B. In general, motilities of the mesenchymal HCCs were higher than those of the epithelial ones. Among them, the mesenchymal phenotype HCC329 and HCC372 exhibited the highest motility, and the epithelial type HCC340 and HCC374 exhibited the lowest motility (FIG. 1C).

### Example 3: Analyzing Signaling in Patient-Derived HCCs

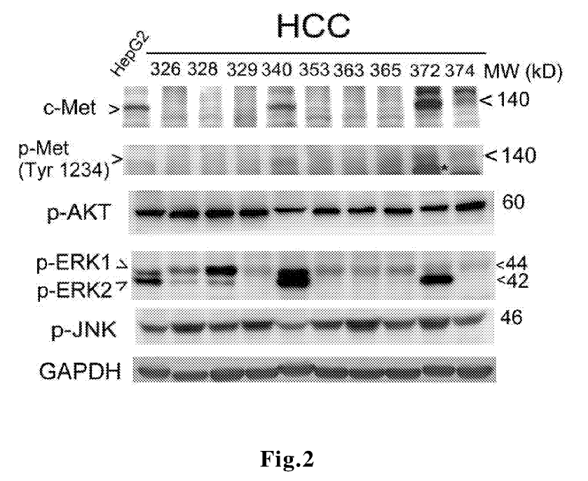

The status of critical signaling components involved in tumor progression of HCCs, including c-Met, ERK, JNK and AKT, were further examined in the patient derived cell lines established in Example 1 and the conventional HCC cell line HepG2. Total proteins were harvested from the cells and subjected to immuno-blotting analysis using GAPDH as a loading control. Antibodies for p-c-Met, p-JNK, p-ERK and p-paxillin (S178), GAPDH and ERK were purchased from Santa Cruz Biotechnology (California, USA). The band intensities on the blots were quantified with Image J. Software. The results shown in FIG. 2 were representative of 2 reproducible experiments.

As demonstrated in FIG. 2, c-Met (β subunit with M.W. 140 kD) was highly expressed in HCC 372, 340 and HepG2, slightly detected in HCC374 but not observed in the other cell lines. It was known that c-Met dimerization activates the phosphorylation of tyrosine residues (Tyr1234) in the kinase domain, which leads to autophosphorylation of the carboxy-terminal substrate binding site (Tyr1349 and Tyr1356) for various cytoplasmic effector proteins. In similar with the pattern of c-Met expression, phosphorylation of c-Met (p-c-Met) at Tyr1234 was detected in HCC372 and HCC340, marginally detected in HepG2 and HCC374 but not observed in the other HCCs. A very intensive band (marked as *) beneath the p-c-Met (Tyr1234) was observed in HCC372, which remains to be identified. In addition, the other two phosphorylated-c-Mets (at Tyr1356 and Tyr1349) were not detected in all of the cell lines (data not shown). In regard with the downstream signaling components, phosphorylated JNK (p-JNK) was very abundant in most of the HCCs, and relatively low in HCC340 and HepG2. On the other hand, the levels of both phosphorylated ERK1 and 2 (p-ERK1 and p-ERK 2) were dramatically higher in HCC340 and relatively higher in HepG2. Also, p-ERK2 (but not p-ERK1) was very high in HCC372 whereas p-ERK1 (but not p-ERK2) was very high and relatively high in HCC328 and HCC326, respectively. In the other HCCs including HCC329, HCC353, HCC363, HCC365 and HCC374, only p-ERK1 can be marginally detected. In addition, abundant p-AKT can be detected in most of the HCCs. In summary, although c-Met signaling was positive in only some of the cell lines (including HCC340, 372, 374 and HepG2) but negative in others (including HCC326, 329, 353, 363, 365), the downstream ERK, JNK and AKT were active in most of the HCCs. Thus, both c-Met dependent and independent signaling existed in these HCCs.

### Example 4: Preparation of FIPs

FIP-gts of SEQ ID NO: 1 was recombinantly expressed in Saccharomyces cerevisiae host cells as described in WO2005/040375A1. Cells expressing FIP-gts were disrupted, centrifuged, and the supernatant was passed through filters and molecular sieves to obtain proteins between 10 kDa and 100 kDa. The filtrate was further purified using FPLC with Superdex® 75 columns (GE Healthcare). The purity was determined to be more than 95% by FPLC.

FIP-fve (SEQ ID NO: 2), FIP-vvo (SEQ ID NO: 3) and FIP-gmi (SEQ ID NO: 6) were prepared using the same method as stated herein, and the proteins thus prepared were found to have a purity of more than 95%.

### Example 5: Suppressive Effects of FIP-gts on Metastatic Phenotype of HCCs

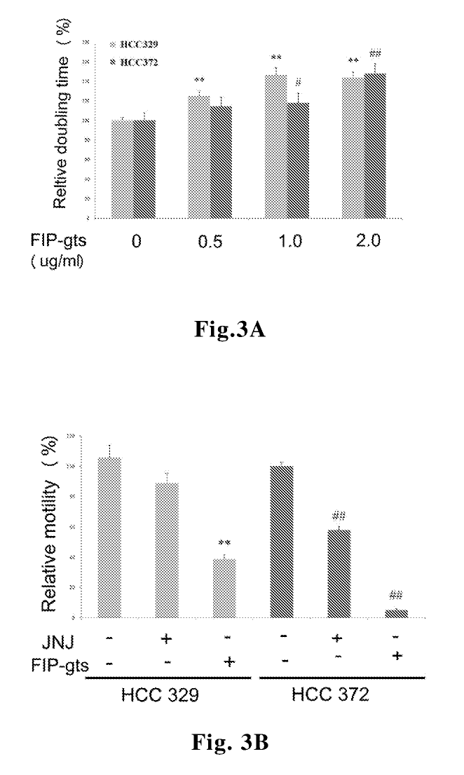

The most motile HCC cell lines identified in Example 2, namely HCC372 and HCC329, were employed for testing the molecular and cellular effects of FIP-gts on HCC, in comparison with those of JNJ-38877605 (abbreviated hereafter as JNJ), which is an ATP-competitive inhibitor for the catalytic activity of c-Met. In the cell proliferation assay, HCC329 and HCC372 cells were untreated or treated with FIP-gts (prepared in Example 4) at 0.5, 1.0 or 2.0 μg/ml for 72 hours. Cell numbers were counted, and the relative doubling times of the respective treated groups were determined by taking the data of the untreated group as 100%. The wound healing migration assay was repeated as described in Example 2, except that HCC329 and HCC372 were additionally treated with FIP-gts and JNJ (MedKoo Biosciences, Chapel Hill, N.C., USA), respectively, for 48 hours. Relative migration was analyzed quantitatively by taking the motility of untreated cells (Con) as 1.0. The results were shown in FIGS. 3A and 3B, in which the symbols (**), (*) and (#) represent statistical significance (p<0.005 and p<0.05, respectively, n=3) between the indicated inhibitor-treated samples and untreated HCC329 or HCC372 control group.

As shown in FIG. 3A, 0.5, 1.0 and 2.0 μg/ml FIP-gts dose dependently increased doubling times of HCC329 and HCC 372 by 25-46% and 14-47%, respectively, demonstrating its anti-proliferative activity on both HCCs.

Further, the effects of FIP-gts on cell migration of both HCC were examined. As demonstrated in FIG. 3B, FIP-gts (2.0 μg/ml) greatly suppressed cell migration of HCC372 by 95%. In parallel, the c-Met specific antagonists JNJ-38877605 (JNJ, at IC50: 26.5 nM) suppressed migration of HCC372 by 50%. On the other hand, FIP-gts and JNJ suppressed cell migration of HCC329 by 80% and 15%, respectively. It is worthy of noting that the extent of inhibition by JNJ was higher in HCC372 than in HCC329. This can be explained by that c-Met signaling was active in HCC372 but not HCC329. In addition, combination of FIP-gts with JNJ didn't show enhanced effects on blocking migration of both HCCs, as compared with treatment of FIP-gts alone (data not shown). Taken together, FIP-gts can suppress constitutive cell migration and cell proliferation of both c-Met positive and negative HCCs.

In a separate experiment, human hepatoma cell line HepG2, a non-motile HCC cell line conventionally used for observing HGF-inducible molecular and cellular effects, was employed. In this experiment, the wound healing migration assay described in Example 2 was again repeated, except that HepG2 (purchased from Bioresource Collection and Research Center (BCRC), Hsinchu, Taiwan) was treated with FIP-gts or JNJ-38877605 for 48 hours in the presence or absence of 25 nM HGF (PeproTech Inc., Rocky Hill, N.J., USA). Relative migration was quantitatively analyzed taking the motility of untreated cells (Con) as 1.0. The results were shown in FIG. 3C, in which the symbol (**) represents statistical significance (p<0.005, n=3) between the indicated HGF-/inhibitor-treated samples and the HGF alone group. Consistent with the data shown in FIG. 2B, FIP-gts (2.0 μg/ml) prevented HGF-induced cell migration of HepG2 much more effectively than JNJ (26.5 nM) (FIG. 3C).

### Example 6: FIP-gts Suppressed Tumor Progression of HCC329 in SCID Mice

The suppressive effects of FIP-gts on tumor development and metastasis of HCC329 were demonstrated in the severe combined immunodeficiency (SCID) mice model. HCC329 cells (approximately 2×10⁷) suspended in 100 μl Dulbecco's Modified Eagle Medium (DMEM; Gibco, Grand Island, N.Y., USA) were directly injected into the subserosa of the middle liver lobe of the SCID mice, followed by intraperitoneal administration twice a week with DMEM as a vehicle, vehicle-containing FIP-gts (4.0-20 μg/g mouse) or JNJ (0.5-2.0 nmole/g mouse).

The mice were sacrificed 2 months after injection. Tumor mass was measured and the occurrence of intrahepatic metastasis and/or extrahepatic metastasis was recorded. Nodules with diameter of more than 0.1-0.2 cm observed on the left or right lobes were recognized as secondary tumor foci. Intra-hepatic metastasis was defined to be the case where at least two secondary tumor foci were observed in the left and/or right liver lobes of the treated mouse. Extra-hepatic metastasis was defined to be the case where the tumors appearing in organs other than liver such as in intestine. During the animal experiment, the regulations relevant to the care and use of laboratory animals were followed. This was approved by the Institutional Animal Care and Use Committee (IACUC) in Tzu Chi University (No. of consent form: 102080).

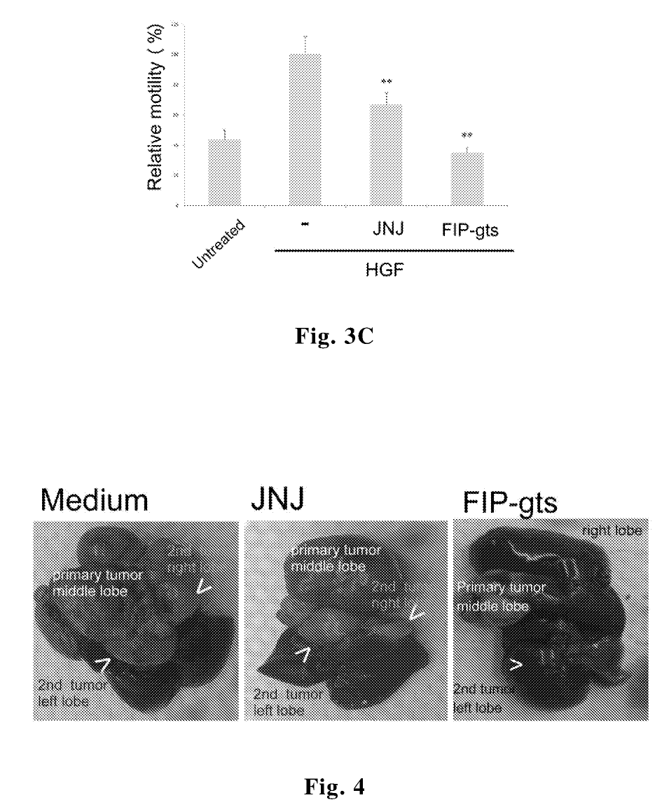

As indicated by the white arrowheads in FIG. 4, secondary tumors were observed on left and right liver lobes in the medium- and JNJ-treated groups, whereas the FIP-gts-treated group shows a normal liver lobule structure, indicating that FIP-gts dramatically suppressed tumor progression, including primary tumor growth and intra-metastasis, of HCC329 in the SCID mice model (Fisher test p<0.05, N=3). In comparison, the effect of c-Met inhibitor JNJ on tumor progression of HCC329 was not prominent (FIG. 4). The toxicity test of FIP-gts on SCID mice was also performed by treating 4 normal SCID mice without inoculation of HCC329. After 2-3 month of intraperitoneal administration of FIP-gts (20 μg/g mouse), no adverse effects was observed in terms of the activity of the animals and the integrities of every organs were preserved (data not shown).

### Example 7: Effects of FIP-gts on c-Met Dependent and Independent Signaling in HCCs

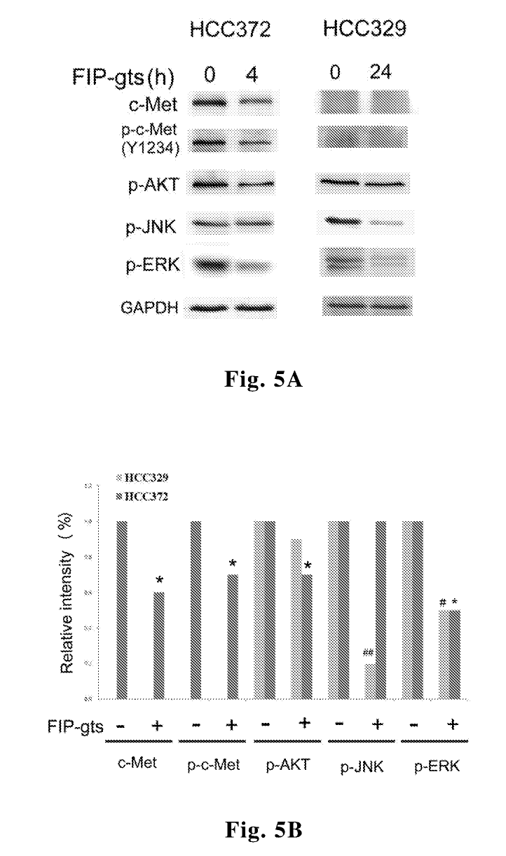

To investigate the molecular mechanism of FIP-gts in suppression of tumor progression of HCCs, the effects of FIP-gts on the activity and/or quantity of the critical members present in c-Met dependent signaling pathway, including c-Met, phosphorylated c-Met (p-c-Met) and downstream effectors p-JNK and p-ERK, were examined in HCCs. HCC329 and HCC372 cells were treated with FIP-gts (2 μg/ml) for 4 or 24 hours. Total proteins were harvested from the cells and subjected to immuno-blotting analysis using GAPDH as a loading control. Antibodies for p-c-Met, p-JNK, p-ERK and p-paxillin (S178), GAPDH and ERK were purchased from Santa Cruz Biotechnology (California, USA). The band intensities on the blots were quantified with Image J. Software. Relative band intensity of each molecule was calculated, taking the data of untreated HCC372 or HCC329 as 1.0. The results were shown in FIGS. 5A and 5B, in which (**) (##) and (*) (#) represent statistical significance (p<0.005 and p<0.05, respectively, n=3) between the indicated inhibitor-treated sample and untreated HCC329 or HCC372 control group.

As demonstrated in FIGS. 5A and 5B, FIP-gts (2.0 μg/ml) decreased phosphorylation of JNK, ERK and AKT after treatment for 24 hours by 85, 51 and 18%, respectively in HCC 329. The effect of FIP-gts on signaling transduction in HCC372 was different from that in HCC329 in several aspects. Firstly, the suppressive effect of FIP-gts can be observed earlier in HCC372 (at 3-4 hours) than in HCC329 (at 24 hours). Secondly, FIP-gts can alter c-Met related signal molecules in the c-Met positive HCC372, which is negative in HCC329. As demonstrated in FIGS. 5A and 5B, expression of Met protein was greatly suppressed by FIP-gts by 90-95% early at the 4th hour in HCC372, which may sustain until 24 hours (not shown). Consistently, FIP-gts significantly suppressed phosphorylation of Met (at Tyr1234) by 55% and greatly suppressed phosphorylation of ERK and AKT by 80-95% at the 4th hour (FIGS. 5A and 5B). In contrast, FIP-gts didn't decrease p-JNK at all in HCC372. Taken together, FIP-gts can suppress activities of critical signal molecules in both c-Met positive and negative HCCs.

Separate studies showed that phosphorylated EGFR (p-EGFR) was remarkably reduced in HCC329 treated with FIP-gts for 16 hours, as compared with that observed in the untreated HCC329 (data not shown). Administration of EGFR inhibitors, including AG1748, and SU5416, suppressed the cell migration of HCC329 by 80% in wound healing assays and reduced the level of p-JNK by 30% within 4 to 16 hours (data not shown). These findings indicate that EGFR-JNK signaling, instead of HGFR signaling, is responsible for mediating cell migration of HCC329, which can be effectively suppressed by the FIP, and that the FIP appears to suppress tumor progression by multiple mechanisms, including by inhibiting the c-Met signaling pathway and by inhibiting the EGFR signaling pathway. This versatility may therefore provide a great clinical advantage, as there is no need to screen HCC patients for c-Met before administration of the FIP to the patients.

### Example 8: Effects of FIP-gts on HGF-Induced c-Met Dependent Signaling in HCC

HepG2 cells were treated with FIP-gts (2.0 μg/ml) for 0.5 hour in the presence or absence of HGF (25 nM). Total proteins were harvested from the cells and immuno-blotting analysis was performed as described in Example 6, except using ERK as the loading control. The results were shown in FIGS. 5C and 5D, in which (**) represent statistical significance (p<0.005, n=3) between the indicated HGF/FIP-gts co-treated sample and the HGF alone group.

As demonstrated in FIG. 5C, FIP-gts greatly suppressed HGF-induced c-Met signaling, including p-c-Met, p-JNK, p-ERK and phosphorylated paxillin (at Ser 178) by 85-90% in HepG2 (FIG. 5D).

Taken together the results shown in Examples 7 and 8, it was found in the present invention that FIP-gts is capable of suppressing both constitutive and HGF-induced c-Met signaling, and also constitutive c-Met independent signaling in HCCs. The results shown in Examples 5 and 7 further indicated that the blockage of c-Met dependent signaling by the FIP is responsible for the reduction in HCC cell migration.

### Example 9: Suppressive Effects of FIP-gmi, FIP-fve and FIP-vvo on HCCs

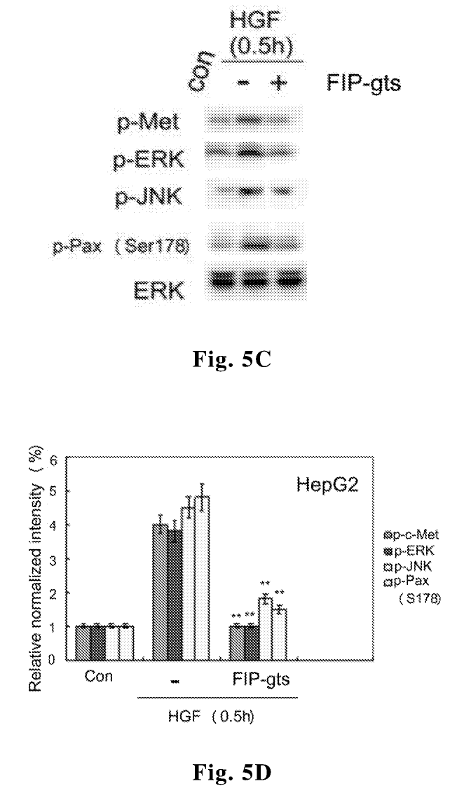

HCC372 and HCC329 cell lines established in Example 2 were cultured in 24-well plates at 1×10⁵ cells/well in Dulbecco's Modified Eagle Medium (DMEM; Sigma, St. Louis, Mo., USA) supplemented with 1% heat-inactivated fetal bovine serum (FBS; Gibco, N.Y., USA) in the presence or absence of FIP-gmi, FIP-fve and FIP-vvo prepared in Example 4 at 1, 2.5 and 5 μg/ml for 48 hours. The supernatant was removed and 25 μl per well of 2.5 mg/ml MTT (3-[4,5-dimethylthiazol-2-yl]-2,5-dipheyltetrazolium bromide; Sigma Chemical Co., St. Louis, Mo., USA) in phosphate-buffered saline (PBS) was added. The plates were incubated at 37° C. for 1 hour to allow the transformation of MTT into water-insoluble formazan crystals. Then, the supernatant was removed and dimethyl sulfoxide (DMSO) was added to all wells and mixed thoroughly to dissolve the dark blue crystals. After a few minutes at room temperature to ensure that all crystals were totally dissolved, the plates were read at 570 nm. Relative survival rate was calculated by taking the number of untreated cells (Con) as 100%. The results are shown in FIGS. 6A and 6B, where the data were representative of 4 reproducible experiments.

The results show that FIP-gmi suppressed the growth of HCC372 by 23% and 96% at 2.5 μg/ml and 5 μg/ml, respectively, and suppressed the growth of HCC329 by 23% and 94% at 2.5 μg/ml and 5 μg/ml, respectively. It further shows that FIP-vvo suppressed the growth of HCC372 by 48% and 96% at 2.5 μg/ml and 5 μg/ml, respectively, and suppressed the growth of HCC329 by 54% and 95% at 2.5 μg/ml and 5 μg/ml, respectively, while FIP-fve suppressed the growth of HCC372 by 13% at 2.5 μg/ml and 5 μg/ml, and suppressed the growth of HCC329 by 18% and 22% at 2.5 μg/ml and 5 μg/ml, respectively. Consistent with the results obtained using FIP-gts as shown in Example 5, FIP-gmi, FIP-fve and FIP-vvo were shown capable of inhibiting cell proliferation of both c-Met positive and negative HCCs.

Further, the effects of the three FIPs on migration of both HCC cells were examined. HCC372 and HCC329 cells were seeded on a 24-well transwell migration insert (Nalge Nunc International Corp., Rochester, N.Y., USA) in a complete medium for 24 hours. After treatment with FIP-gmi (3 μg/ml), FIP-fve (10 μg/ml) and FIP-vvo (2.5 μg/ml) for 48 hours, cells that had migrated to the underside of the insert membrane were stained with 0.3% crystal violet. The cells on the topside of the insert membrane were rubbed with a cotton swab. The migrated cells on the underside were imaged using phase contrast microscopy with 200× magnification.

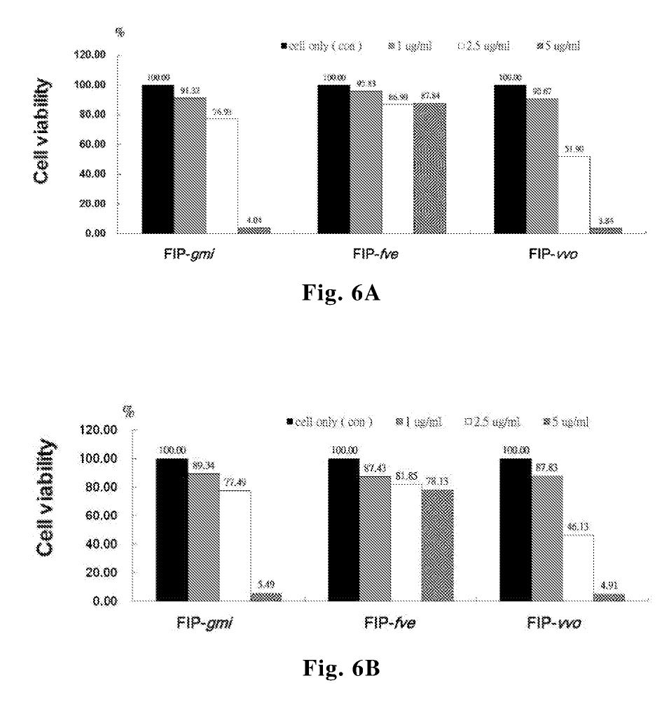

As demonstrated in FIG. 7, FIP-gmi and FIP-vvo dramatically suppressed cell migration of HCC329 and HCC372 by 95-99%, whereas FIP-fve marginally suppressed cell migration of HCC372 and HCC329. Consistent with the results of FIP-gts shown in Example 5, FIP-gmi, FIP-fve and FIP-vvo were shown capable of inhibiting migration of both c-Met positive and negative HCC cells.

### Example 10: Effects of FIP-gmi, FIP-fve and FIP-vvo on c-Met Dependent Signaling in HCCs

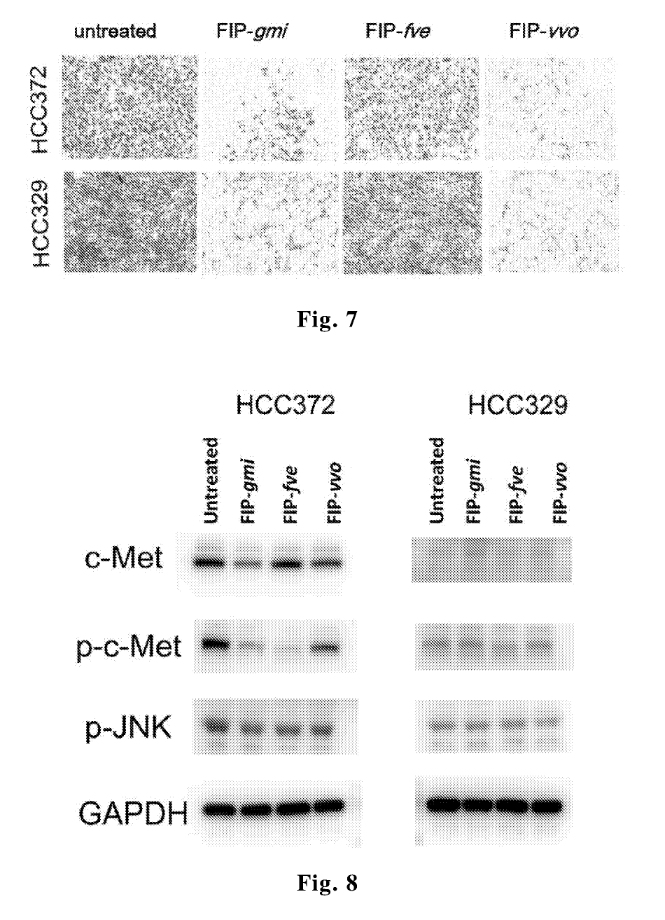

The experiments described in Example 7 were repeated, except that FIP-gts was replaced with FIP-gmi, FIP-fve and FIP-vvo for treating HCC329 and HCC372. The results of the western blotting analysis were shown in FIG. 8. Consistent with the results obtained using FIP-gts as shown in Example 7, c-Met, p-c-Met and p-JNK were shown to be significantly suppressed by FIP-gmi, FIP-fve and FIP-vvo in the HGFR-positive HCC cell line, HCC372, suggesting that the three FIPs, like FIP-gts, perform inhibitory activity on c-Met dependent signaling and, as a consequence, may suppress cell migration and metastasis of HCC372 via blocking c-Met dependent signaling. In addition, since c-Met was not substantially expressed in HCC329, the inhibitory effects of FIP-gmi, FIP-fve and FIP-vvo on HCC329 must be exerted through blockage on a c-Met independent signaling, such as EGFR-dependent signaling pathway.

While the invention has been described with reference to the preferred embodiments above, it should be recognized that the preferred embodiments are given for the purpose of illustration only and are not intended to limit the scope of the present invention and that various modifications and changes, which will be apparent to those skilled in the relevant art, may be made without departing from the spirit and scope of the invention.

All papers, publications, literature, patents, patent applications, websites, and other printed or electronic documents referred herein, including but not limited to the references listed below, are incorporated by reference in their entirety. In case of conflict, the present description, including definitions, will prevail.

What is claimed is:

1. A method for inhibiting hepatocyte growth factor receptor (HGFR) activity in a cell, comprising contacting an effective amount of a fungal immunomodulatory protein (FIP) with the cell.

2. The method according to claim 1, wherein the cell is derived from an HGFR-associated cancer selected from the group consisting of hepatocellular carcinoma, hereditary and sporadic human papillary renal carcinomas, ovarian cancer, prostate carcinoma, gallbladder carcinoma, breast carcinoma, melanoma, glioblastoma, head-and-neck squamous carcinoma, esophageal cancer, gastric cancer, pancreatic cancer, mesothelioma, colorectal cancer, osteogenic sarcoma, lymphoma and multiple myeloma.

3. (canceled)

4. (canceled)

5. (canceled)

6. (canceled)

7. A method for inhibiting metastasis of hepatocellular carcinoma in a subject, comprising administering to said subject an effective amount of a fungal immunomodulatory protein (FIP).

8. A method for preventing recurrence of hepatocellular carcinoma in a subject, comprising administering to said subject an effective amount of a fungal immunomodulatory protein (FIP).

9. The method according to claim 7 or 8, wherein the hepatocellular carcinoma is an HGFR-positive hepatocellular carcinoma.

10. The method according to claim 7 or 8, wherein the hepatocellular carcinoma is an HGFR-negative hepatocellular carcinoma.

11. The method according to anyone of claims 1, 7 and 8, wherein the FIP has an amino acid homology of at least 57% to the amino acid sequence of SEQ ID NO: 1.

12. The method according to claim 11, wherein the FIP has an amino acid sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7 and SEQ ID NO: 8.

13. The method according to claim 12, wherein the FIP has an amino acid sequence of SEQ ID NO: 1.

14. The method according to anyone of claims 1, 7 and 8, wherein the FIP is in an orally administrable form.

15. The method according to anyone of claims 1, 7 and 8, wherein the FIP is in a parenteralyl administrable form.

16. (canceled)

17. (canceled)

18. The method according to anyone of claims 1, 7 and 8, wherein the subject is selected from the group consisting of human and non-human vertebrates.

19. (canceled)
